(19) United States
(12) Patent Application Publication
(10) Pub. No.: US 2017/0145515 A1
(43) Pub. Date: May 25, 2017

(54) MOLECULAR MAMMOGRAPHY

(71) Applicant: ATOSSA GENETICS INC., Seattle, WA (US)

(72) Inventors: Shu-Chih CHEN, Seattle, WA (US); Steven C. QUAY, Seattle, WA (US)

(21) Appl. No.: 15/316,136

(22) PCT Filed: Jun. 2, 2015
(86) PCT No.: PCT/US15/33827

§ 371 Date: Dec. 2, 2016

Related U.S. Application Data
(60) Provisional application No. 62/007,830, filed on Jun. 4, 2014.

(51) Int. Cl. C12Q 1/68

(52) U.S. Cl. CPC C12Q 1/6886; C12Q 2600/112; C12Q 2600/178; C12Q 2600/154; C12Q 2600/118; C12Q 2600/158

(57) ABSTRACT

The disclosure relates to methods of screening and diagnosing cancer in patients undergoing mammography.

### CROSS-REFERENCE

This application claims the benefit of U.S. Provisional Patent Application No. 62/007,830, filed Jun. 4, 2014, which is incorporated by reference herein in its entirety.

### BACKGROUND OF THE INVENTION

Breast cancer is by far the most common form of cancer in women, and it is the second leading cause of cancer death in humans. Despite advances in diagnosing and treating breast cancer, the prevalence of this disease has been steadily rising at a rate of about 1% per year since 1940. Today, the likelihood that a women living in North America will develop breast cancer during her lifetime is one in eight.

The current widespread use of mammography has resulted in improved detection of breast cancer. Nonetheless, the death rate due to breast cancer has remained unchanged at about 27 deaths per 100,000 women. Breast Imaging-Reporting and Data System (BI-RADS), a widely accepted predictive value risk assessment and quality assurance tool in mammography, is used by physicians to classify breast lesions into several BI-RADS categories, ranging from 0 to VI and to make diagnostic and disease management recommendations based on BI-RADS classification. All too often, breast cancer is discovered at a stage that is too far advanced, when therapeutic options and survival rates are severely limited, likely to due to a higher degree of erroneous assignment to incorrect BI-RADS categories and its influence on clinical factors. In fact, the information that women get based on their BI-RADS classification can mean a difference between being recalled or not recalled for further assessment and a potential for failure of early detection. While there is a high level of concordance or agreement amongst radiologists on lower end of the scale (i.e., BI-RADS I or II) and on the higher end of the scale (BI-RADS IV or V), there appears to be higher degree of discordance observed between radiologists in their assignment of breast lesions into categories II and III. Inter-and intra-radiologist variability in the BI-RADS assessment and breast density groups categories for screening mammograms. Redondo et al. Br J Radiol. 85(1019); 2012 Nov, pages 1465-1470; Use of the American College of Radiology BI-RADS guidelines by community radiologists: concordance of assessments and recommendations assigned to screening mammograms. Lehman et al. Am. J. Roentgenology. 2002; 179(1), pages 15-20. Thus, there is an unmet need for better disease classification, early detection of breast lesions, and diagnosis of breast disorders.

### SUMMARY OF THE INVENTION

Disclosed herein, in certain embodiments, are methods of diagnosing or prognosing a breast disorder in an individual in need thereof, comprising screening intraductal fluid obtained from a nipple of the individual during mammography for at least one biomarker associated with a breast disorder. In certain embodiments, the methods of diagnosing or prognosing a breast disorder in an individual need thereof are particularly useful for the diagnosis or prognosis of individual having a BI-RADS III or a BI-RADS IV lesion. In some embodiments, the methods further comprise contacting the nipple with a collection device. In some embodiments, the collection device comprises a solid phase sample collection medium. In some embodiments, the collection device further comprises a breast engaging member which attaches the device to the breast. In some embodiments, the solid phase sample collection medium is selected from absorbent paper, microscopic glass slides, capillary tubes, collection tubes, columns, micro-columns, wells, plates, membranes, filters, resins, inorganic matrices, beads, particulate chromatographic media, plastic microparticles, latex particles, coated tubes, coated templates, coated beads, coated matrices, or a combination thereof. In some embodiments, the methods further comprise removing keratin from nipple ducts of the breast of the individual prior to the mammography. In some embodiments, the methods further comprise administering atropine to the nipple of the individual prior to the mammography. In some embodiments, the methods further comprise administering oxytocin to the individual prior to performing mammography. In some embodiments, the screening comprises contacting a cell from the intraductal fluid with an antibody that binds to an antigen selected from the group consisting of: CK5, CK14, CK7, CK18, and p63. In some embodiments, the at least one biomarker comprises cytology, proteins, glycoproteins, DNA, RNA, gene mutations, single nucleotide polymorphisms, DNA copy numbers, methylation of DNA, histone, and/or proteins, microRNA, microbiome or a combination thereof. In some embodiments, the screening comprises contacting a cell from the intraductal fluid with an antibody that binds to an antigen selected from the group consisting of: CK5, CK14, CK7, CK18, and p63. In some embodiments, the screening comprises determining a presence and/or a level of one or more miRNA, profiling miRNA signature, or a combination thereof in the intraductal fluid sample. In some embodiments, the miRNA is selected from Table 3, Table 4, Table 5, or a combination thereof. In some embodiments, the miRNA in the intraductal fluid sample is exosomal. In some embodiments, the screening comprises amplification, sequencing, restriction length polymorphism analysis, microarray analysis, multiplex analysis, or a combination thereof. In some embodiments, the screening comprises contacting a cell from the intraductal fluid with antibodies that bind to uPA, PAI-1, and Gal-GalNAc, detecting in the intraductal fluid sample altered miRNA signature disclosed in Table 5, or detecting in the intraductal fluid sample an altered DNA methylation pattern of uPA, PAI-1 and GalNac Transferases genes.

In some embodiments, the methods further comprise determining or modifying a treatment regimen for the individual based on results of the screening. In some embodiments, the treatment regimen comprises a therapeutic agent, radiation therapy, and/or surgical excision of breast tissue. In some embodiments, the therapeutic agent is an anthracycline (e.g., doxorubicin or epirubicin), a platinum agent, a taxane (e.g., paclitaxel or docetaxel), or a combination thereof. In some embodiments, the therapeutic agent is ado-trastuzumab emtansine, albumin-bound paclitaxel, anastrozole, butyric acid, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen, or a tamoxifen derivative (such as 4-hydroxytamoxifen, N-desmethyltamoxifen and cis-tamoxifen), toremifene, trastuzumab, vinorelbine, or a combination thereof. In some embodiments, the thereapeutic agent is a ado-trastuzumab emtansine, albumin-bound paclitaxel, anastrozole, butyric acid, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen, tamoxifen derivative, 4-hydroxytamoxifen, N-desmethyltamoxifen, endoxyfen, cis-tamoxifen, toremifene, trastuzumab, vinorelbine, or a combination thereof. In some embodiments, the therapeutic agent is a SERM, a SERD, an AI, a pharmaceutical salt thereof, or a combination thereof. In some embodiments, the therapeutic agent further comprises at least one omega-3 fatty acid, and at least one vitamin D compound.

### DETAILED DESCRIPTION OF THE INVENTION

Disclosed herein, in certain embodiments, are methods of diagnosing or prognosing a breast disorder in an individual in need thereof comprising screening a sample of intraductal fluid obtained during mammography of a breast of the individual for markers of a breast disorder. In some embodiments, the sample of intraductal fluid comprises mammary fluid, whole cells, cell fragments, cell membranes, selected liquid, cellular or other solid fractions of the intraductal fluid, as well as proteins, glycoproteins, peptides, lipids, sugars, oligosaccharides, glycolipids, nucleotides (including DNA and RNA polynucleotides) and other like biochemical and molecular constituents of the intraductal fluid. In some embodiments, the methods comprise contacting a collection device with the breast. In some embodiments, pressure exerted by a mammography device results in expression of intraductal fluid from the breast. In some embodiments, the sample of intraductal fluid is collected by the collection device. In some embodiments, the sample of intraductal fluid is screened for biomarkers of a breast disorder.

### DEFINITIONS

“The terms “individual,” “subject,” or “patient” are used interchangeably. As used herein, they mean any mammal (i.e., species of any orders, families, and genus within the taxonomic classification animalia: chordata: vertebrata: mammalia). In some embodiments, the mammal is a human None of the terms require or are limited to situation characterized by the supervision (e.g. constant or intermittent) of a health care worker (e.g., a doctor, a registered nurse, a nurse practitioner, a physician's assistant, an orderly, or a hospice worker).

As used herein, “breast disorder” means any disorder of a breast. Breast disorders include benign lesions of the breast and breast cancer. Benign breast lesions include, but are not limited to, dense breast, mastitis, columnar cell hyperplasia, columnar cell hyperlasia with atypia, ductal hyperplasia, lobular hyperplasia, atypical ductal hyperplasia, and atypical lobular hyperplasia.

As used herein, “breast cancer” means any malignant tumor of breast cells. There are several types of breast cancer. Exemplary breast cancers include, but are not limited to, ductal carcinoma in situ, lobular carcinoma in situ, invasive (or infiltrating) ductal carcinoma, invasive (or infiltrating) lobular carcinoma, inflammatory breast cancer, triple-negative breast cancer, ER+ breast cancer, HER2+ breast cancer, adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, and micropapillary carcinoma. A single breast tumor can be a combination of these types or be a mixture of invasive and in situ cancer.

The term “diagnosis” as used herein refers to the identification of a molecular or pathological state, disease or condition, such as the identification of a breast disorder or a molecular subtype of breast disorder.

The term “prognosis” as used herein refers to the prediction of the likelihood of breast cancer-attributable death or progression, including recurrence, metastatic spread, and drug resistance, of a breast disorder. The term “prediction” may refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

### Current Methods of Diagnosis

A mammogram is an x-ray of a breast. It uses ionizing radiation to create images of breast tissue that enable visualization of masses and/or microcalcifications. Mammograms are used to check for breast disorders in women who have no signs or symptoms of disease and to check for breast disorders after a lump or other sign or symptom of disease is found.

Mammography reduces the number of deaths from cancer among women ages 40 to 74. However, mammography has several drawbacks, including: false-positive results and over-diagnosis, false-negative results and under diagnosis, and radiation exposure. False-positive results occur when a radiologist incorrectly interprets a mammogram as indicating that breast cancer is present when it is not. False-positive results lead to over-diagnosis and overtreatment. Studies have shown the chances of having a false positive result after 10 yearly mammograms are about 50 to 60 percent. False-positives are particularly common where a mammogram reveals ductal carcinoma in situ (DCIS, a noninvasive tumor in which abnormal cells that may become cancerous build up in the lining of breast ducts). This leads to over-diagnosis and overtreatment. False-negative results lead to under-diagnosis and advancement of the cancer. False-negative results are common in individuals who have dense breasts or have lobular, mucinous or rapidly growing cancers. Most pre-menopausal women have dense breasts, and many post-menopausal women also have dense breasts. Mammography can have a sensitivity for picking up cancer in a women with dense breast to 15% to 30%.

A widely accepted predictive value risk assessment and quality assurance tool in mammography is the Breast Imaging-Reporting and Data System (BI-RADS). Breast lesions are classified into several BI-RADS categories, ranging from 0 to VI. Physicians make recommendations using BI-RADS as a breast disorder management tool.

TABLE 1
BI-RADS Assessment Categories and Management Recommendations

| BI-RADS | | | Likelihood of |
|---|---|---|---|
| Category | Lesions Status | Management | Cancer/Malignancy |
| 0 | Incomplete | Recall for additional | N/A |
| | | imaging/comparison | |
| | | with prior examination | |
| I | Negative | Routine mammography | Essentially 0% |
| | | screening | |
| II | Benign | Routine mammography | Essentially 0% |
| | | screening | |
| III | Probably benign | Short-interval | 0%-≦2% |
| | | screening (6 m) follow | |
| | | up or continued | |
| | | surveillance | |
| | | mammography | |
| IV | Suspicious | Tissue diagnosis | >2%-≦95% |
| IVa | abnormality | | |
| IVb | Low | | >2%-≦10% |
| IVc | suspicion for | | |
| | malignancy | | |
| | Moderate | | >10%-≦50% |
| | suspicion for | | |
| | malignancy | | |
| | High | | >50%-≦95% |
| | suspicion for | | |
| | malignancy | | |
| V | Highly suggestive | Tissue diagnosis | ≧95% |
| | of malignancy | | |
| VI | Proven | Surgical excision when | N/A |
| | malignancy | clinically appropriate | |

While high level of interobserver concordance on lower end of the scale (i.e., BI-RADS I or II) and on the higher end of the scale (BI-RADS IV or V), greater level of lack of concordance (i.e., discordance) in interobserver has been reported between BI-RADS categories II and III (Inter-and intra-radiologist variability in the BI-RADS assessment and breast density groups categories for screening mammograms. Redondo et al. Br J Radiol. 85(1019); 2012 Nov, pages 1465-1470; Use of the American College of Radiology BI-RADS guidelines by community radiologists: concordance of assessments and recommendations assigned to screening mammograms. Lehman et al. Am. J. Roentgenology. 2002; 179(1). pages 15-20). The assessment with the highest discordance was “probably benign finding” (category III), at 53.5%. For example, a disagreement between radiologists regarding classification into BI-RADS categories II and III or into categories IVa and IVb means that one of the radiologists has detected a benign lesion and finds no reason to recall the individual for further assessment and the other has found a probably benign lesion and recommends further assessment and this seems to be a subjective evaluation (Redondo et al.; BI-RADS Lexicon for US and Mammography: Interobserver variability and Positive Predictive Value. Lazarus et al. Radiology. 2006, 239(2), pages 385-391). In fact, the information that women get, which is what modifies the next step, is recall for further assessment. But there has been some tension in the radiologist community with regards to the assignment of final assessment BI-RADS categories III and IV due to a higher degree of erroneous assignment and its influence on clinical factors. Thus, there is unmet need for better disease classification, early detection of breast lesions and diagnosis of breast disorders.

### Breast Disorders

The normal breast consists of ducts and lobules with a dual-layered architecture. Luminal secretory cells surround a hollow lumen, and in turn are surrounded by a layer of myoepithelial cells that lie in direct contact with the basement membrane.

### Breast Hyperplasia

Hyperplasia (also known as epithelial hyperplasia or proliferative breast disease) is an overgrowth of the cells that line either the ducts or the lobules. When hyperplasia is in the duct, it is called ductal hyperplasia or duct epithelial hyperplasia. When it affects the lobule, it is referred to as lobular hyperplasia.

Hyperplasia is usually diagnosed with a core needle biopsy or surgical biopsy. Based on how the cells look under the microscope, hyperplasia is characterized as mild hyperplasia, usual hyperplasia, or atypical hyperplasia. Mild hyperplasia does not increase the risk for breast cancer. Hyperplasia of the usual type (without atypia), also known as usual hyperplasia increases the risk of cancer to about 1½ to 2 times that of a woman with no breast abnormalities. Atypical hyperplasia (either atypical ductal hyperplasia [ADH] or atypical lobular hyperplasia [ALH]) increases the risk of breast cancer to about 4 to 5 times higher than that of a woman with no breast abnormalities.

In addition, with the widespread adoption of screening mammography columnar cell lesions (CCL) of the breast including columnar cell hyperplasia (CCH) have become a frequent finding in breast biopsies. Presence of CCL near known precancerous and cancerous changes suggest that CCL may be premalignant and high frequency occurrence of CCL and low grade DCIS are known to be present in the same breast with CCL and DCIS commonly occurring in the same or adjacent terminal duct lobular unit (TDLU). Due to the similarities in cytological and architectural changes in the more advanced CCL and atypical hyperplasia and DCIS, CCL is proposed to be precursor to atypical breast proliferation and breast cancer.

### Breast Cancers

Breast cancer usually begins either in the cells of the lobules or the ducts. A breast cancer may be a “mixed tumor,” meaning that it contains a mixture of cancerous ductal cells and lobular cells. In such cases, the cancer is treated as a ductal carcinoma. If there is more than one tumor in the breast, the breast cancer is described as either multifocal or multicentric. In multifocal breast cancer, all of the tumors arise from the original tumor, and they are usually in the same section of the breast. If the cancer is multicentric, it means that all of the tumors formed separately, and they are often in different areas of the breast.

Invasive vs. Non-Invasive: Addressing a Current Drawback in Mammography

Non-invasive cancers stay within the ducts or lobules in the breast. They do not grow into or invade normal tissues within or beyond the breast. Non-invasive cancers are sometimes called carcinoma in situ (“in the same place”) and many consider them pre-cancers.

Invasive cancers expand or migrate into normal, healthy tissues. Most breast cancers are invasive. Whether the cancer is non-invasive or invasive will affect treatment choices and responses thereto.

A breast cancer may be both invasive and non-invasive. This means that part of the cancer has grown into normal tissue and part of the cancer has stayed inside the milk ducts or milk lobules. In such cases, these cancers would be treated as an invasive.

In most cases, a breast cancer is classified as one of the following: DCIS (Ductal Carcinoma In Situ); MIC (Microinvasive breast carcinoma); MICB and DCIM. DCIS is a non-invasive cancer that stays inside the milk duct. MIC is a subtype of DCIS. It has a size that is less than 1.0 mm and about 10% or less of MIC cells have left the duct tissue (the original tumor site).

LCIS (Lobular Carcinoma In Situ): LCIS is an overgrowth of cells that stay inside the lobule. It indicates an increased risk for developing an invasive cancer. IDC (Invasive Ductal Carcinoma) is the most common type of breast cancer. Invasive ductal carcinoma (IDC) begins in the milk duct but has grown into the surrounding normal tissue inside the breast. ILC (Invasive Lobular Carcinoma) starts inside the lobule but grows into the surrounding normal tissue inside the breast.

TABLE 2
Prevalence and Tumor Characteristics of Different Types and
Special Forms of Invasive Breast Cancer

| Types of | Proportion of | | |
|---|---|---|---|
| invasive | all invasive | Tumor | |
| breast cancer | breast cancers | characteristics | Prognosis |
| Invasive | 50-75% | Hard | Prognosis varies with stage |
| ductal | | tumor texture | and grade of tumor |
| carcinoma | | Tumor is | |
| (IDC) | | irregular, star- | |
| | | shaped | |
| | | Cell features | |
| | | vary | |
| | | DCIS often | |
| | | present | |
| Invasive | 10-15% | Normal, | Prognosis varies with stage |
| lobular | | slightly firm or | and grade of tumor |
| carcinoma | | hard | For any given stage or |
| (ILC ) | | tumor texture | grade, prognosis is similar |
| | | Cells appear in | to that of IDC |
| | | single file order | Pattern of metastases is |
| | | Tumors | slightly different from IDC |
| | | are most | (more likely to go to the |
| | | often ER- | gastrointestinal tract) |
| | | positive and | |
| | | HER2/neu- | |
| | | negative | |
| Medullary | 1-5% | Soft tumor | More common among |
| carcinoma | | Cells have a | younger women and |
| | | sheet-like | women with a BRCA1 |
| | | appearance | genetic mutation |
| | | Tumors are | At this time, it is not |
| | | often ER- | known whether prognosis |
| | | negative | is better than or similar |
| | | | to that for IDC and ILC |
| Mucinous | 1-5% | Soft tumor | More common among |
| (colloid) | | Often no | older women |
| carcinoma | | palpable tumor | Tends to have a |
| | | Cells are | good prognosis |
| | | surrounded by | Less common for cancer to |
| | | excess mucous | spread to lymph nodes |
| | | (mucin) | |
| | | Tumors | |
| | | are most | |
| | | often ER- | |
| | | positive and | |
| | | HER2/neu- | |
| | | negative | |
| Papillary | 1-5% | Soft tumor | More common among |
| carcinoma | | Cells appear as | postmenopausal women |
| | | fingerlike | Tends to have a good |
| | | branches | prognosis |
| Tubular | 1-5%* | Tumors are | Prognosis is usually better |
| carcinoma | | most often | than IDC (survival at 5 |
| | | small | years is 88%) |
| | | Often no | Rare for cancer to spread |
| | | palpable tumor | to lymph nodes or other |
| | | Cells form | parts of the body |
| | | tube-like | |
| | | structures | |
| | | Tumors | |
| | | are most | |
| | | often ER- | |
| | | positive and | |
| | | HER2/neu- | |
| | | negative | |

### Molecular Subtypes

Gene expression profiling classifies breast cancers into four major biologically distinct intrinsic subtypes: luminal A, luminal B, human epidermal growth factor receptor-2 (HER2) over-expressing, and basal-like/triple negative. These molecular subtypes have prognostic and predictive value. The prognosis and chemotherapy sensitivity of the different molecular subgroups are different.

ER+ breast cancer is characterized by the presence of estrogen receptors on the surface of the cancerous cells. Growth of ER+ cancer cells is associated with the availability of estrogen. Treatment options for ER+ breast cancer chemotherapeutic agents that block estrogen (e.g. tamoxifen).

HER2+ breast cancers are characterized by an excess of HER2 on the cell surface of the cancerous cells. HER2+ cancer is often treated with trastuzumab in combination with additional chemotherapeutic agents.

Triple-negative breast cancer is a breast cancer characterized by cells which lack estrogen receptors and progesterone receptors, and do not have an excess of the HER2 protein on their surfaces. Triple-negative breast cancers are often more invasive than other breast cancers. Because the tumor cells lack estrogen and progesterone receptors, hormone therapy (e.g., tamoxifen) is not effective. Additionally, as the cells lack the HER2 protein, drugs that target HER2 (e.g., trastuzumab) are ineffective.

### Luminal Cancers

Most breast cancers are luminal tumors. Luminal tumor cells look like the cells of breast cancers that start in the inner (luminal) cells lining the mammary ducts.

Luminal A breast cancers are ER+ and/or PR+, HER2−, low Ki67. About 42-59% of breast cancers are luminal A. Luminal A tumors tend to be of low or moderate tumor grade. Of the four subtypes, luminal A tumors tend to have the best prognosis, with fairly high survival rates and fairly low recurrence rates. Only about 15% of luminal A tumors have p53 mutations, a factor linked with a poorer prognosis.

Luminal B breast cancers are ER+ and/or PR+, HER2+(or HER2-with high Ki67). About 6-17% of breast cancers are luminal B. Women with luminal B tumors are often diagnosed at a younger age than those with luminal A tumors. Compared to luminal A tumors, luminal B tumors also tend to have factors that lead to a poorer prognosis including: poorer tumor grade; larger tumor size; and p53 gene mutations. In general, women with luminal B tumors have fairly high survival rates, although not as high as those with luminal A tumors.

### Basal-Like

Approximately 14-20% of breast cancers are basal-like. Basal-like breast cancers differ to luminal cancers in being triple negative for the immunophenotypic markers ER-/PR-/HER2-but express CK5/6. Basal-like breast cancers show increased hypoxia and high tumor grade and have an aggressive phenotype characterized by high cell proliferation and poor clinical outcome. Most BRCA1 breast cancers and many BRCA2 breast cancers are both triple negative/basal-like. Triple negative/basal-like tumors are often aggressive and have a poorer prognosis compared to the estrogen receptor-positive subtypes (luminal A and luminal B tumors). Triple negative/basal-like tumors are usually treated with some combination of surgery, radiation therapy and chemotherapy. These tumors cannot be treated with hormone therapies or trastuzumab (Herceptin®) because they are hormone receptor-negative and HER2/neu-negative.

### Method of Diagnosing or Prognosing Breast Disorders

Disclosed herein, in certain embodiments, are methods of diagnosing or prognosing a breast disorder in an individual in need thereof comprising screening a sample of intraductal fluid obtained during mammography of a breast of the individual for markers of a breast disorder. These methods of diagnosing or prognosing a breast disorder in an individual in need thereof are particularly useful for the diagnosis or prognosis of individuals having BI-RADS II-V lesions, preferably BI-RADS II-IV lesions, and even more preferably BI-RADS III-IV lesions. In some embodiments, the methods are useful for distinguishing between luminal and basal breast cancers. In other embodiments, the methods are useful for distinguishing between pre-cancers and cancers, between hyperplasia and cancer, and between invasive and non-invasive cancers. In some embodiments, the sample of intraductal fluid comprises mammary fluid, whole cells, cell fragments, cell membranes, selected liquid, cellular or other solid fractions of the intraductal fluid, as well as proteins, glycoproteins, peptides, lipids, sugars, oligosaccharides, glycolipids, nucleotides (including cell-bound and cell-free DNA (e.g., cfDNA, mitochondrial DNA) and cell-bound and cell-free RNA polynucleotides, cell-free DNA and cell-free RNA, (e.g., mRNA, mitochondrial RNA, and microRNA) and other like biochemical and molecular constituents of the intraductal fluid. In some embodiments, the methods comprise contacting a collection device with the breast. In some embodiments, pressure exerted by a mammography device results in expression of intraductal fluid from the breast. In some embodiments, the amount of fluid expressed is less than 1 microliter. In some embodiments, the amount of fluid expressed is less than 1 nanoliter. In some embodiments, the amount of fluid expressed is between 1 nanoliter and 1 picoliter. In some embodiments, the amount of fluid expressed is 1 picoliter, 2 picoliters, 3 picoliters, 4 picoliters, 5 picoliters, 6 picoliters, 7 picoliters, 8 picoliters, 9 picoliters, 10 picoliters, between 10 and 15 picoliters, or between 15 and 20 picoliters. In some embodiments, the sample of intraductal fluid is collected by the collection device. In some embodiments, the sample of intraductal fluid is screened for biomarkers of a breast disorder. In some embodiments, the methods further comprise cleaning a nipple of the breast. In some embodiments, the methods further comprise administering oxytocin to the individual before the mammography.

### Collection Device

Disclosed herein, in certain embodiments, are methods of diagnosing or prognosing a breast disorder in an individual in need thereof comprising screening a sample of intraductal fluid obtained during mammography of a breast of the individual for markers of a breast disorder. In some embodiments, the methods comprise contacting a collection device with the breast. In some embodiments, the sample of intraductal fluid is collected by the collection device. In some embodiments, the collection device is wearable.

In some embodiments, the collection device comprises a solid phase sample collection medium. In some embodiments, the collection device further comprises a breast engaging member which attaches the device to the breast.

In some embodiments, the solid phase sample collection medium is an absorbent paper. In some embodiments, the absorbent paper absorbs fluids. In some embodiments, the absorbent paper binds to proteins. In some embodiments, the absorbent paper binds to nucleotides, polynucleotides, DNA, RNA or a combination thereof. In some embodiments, the absorbent paper does not bind to cells.

In some embodiments, the collection device comprises absorbent paper. In some embodiments, the absorbent paper absorbs fluids. In some embodiments, the absorbent paper binds to proteins. In some embodiments, the absorbent paper binds to nucleotides, polynucleotides, DNA, RNA or a combination thereof. In some embodiments, the absorbent paper does not bind to cells.

Absorbent papers (which may also be called “membranes” herein) for use with the methods disclosed herein are made of any material suitable for the collection of epithelial cells and biomarkers such as, for example, proteins, carbohydrates, lipids, nucleic acids, RNA, DNA, etc. Absorbent papers include those made of, for example, nitrocellulose, microcellulose, mixed cellulose ester, or any other appropriate material for intraductal fluid sample collection.

In some embodiments, the absorbent paper does not cause papers cuts to the nipple and/or the areola. In some embodiments, the absorbent paper is shaped to avoid paper cuts to the nipple and/or areola.

The absorbent paper is formed by stamping the paper out of large paper stock with a metal mold. The absorbent paper is big enough to cover or partially cover the nipple. In some embodiments, the absorbent paper is big enough to cover the nipple. Therefore, an absorbent paper may be from about 1.0 inches to about 3.0 inches in diameter or length at its average dimension A across any size of the absorbent paper. An absorbent paper may be, for example, about 1.0, about 1.1, about 1.15, about 1.2, about 1.25, about 1.3, about 1.35, about 1.4, about 1.45, about 1.5, about 1.55, about 1.6, about 1.65, about 1.7, about 1.75, about 1.8, about 1.85, about 1.9, about 1.95, about 2.0, about 2.1, about 2.15, about 2.2, about 2.25, about 2.3, about 2.35, about 2.4, about 2.45, about 2.5, about 2.55, about 2.6, about 2.65, about 2.7, about 2.75, about 2.8, about 2.85, about 2.9, about 2.95, or about 3.0 inches in diameter. In some embodiments, the absorbent paper covers or partially covers the areola of a breast. In some embodiments, the absorbent paper covers the areola of a breast. In some embodiments, the absorbent paper partially covers the areola of a breast. In some embodiments, the absorbent paper covers a nipple and does not extend to the areola of a breast.

The thickness of the absorbent paper may vary to allow for optimal sample collection and includes materials that are from about 0.0001 inches to about 0.1 inches in thickness. For example, the absorbent paper may be about 0.0001, about 0.02, about 0.03, about 0.04, about 0.05, about 0.06, about 0.07, about 0.08 about 0.09, or about 0.1 inches thick.

In some embodiments, the solid phase sample collection medium comprises microscopic glass slides, capillary tubes, collection tubes, columns, micro-columns, wells, plates, membranes, filters, resins, inorganic matrices, beads, particulate chromatographic media, plastic microparticles, latex particles, coated tubes, coated templates, coated beads, coated matrices, or a combination thereof.

### Oxytocin

Disclosed herein, in certain embodiments, are methods of diagnosing or prognosing a breast disorder in an individual in need thereof comprising screening a sample of intraductal fluid obtained during mammography of a breast of the individual for markers of a breast disorder. In some embodiments, the methods further comprise administering oxytocin, or an analogue thereof, including carbetocin. to the individual prior to mammography. In some embodiments, the methods further comprise administering carbetocin to the individual prior to mammography.

In some embodiments, oxytocin, or an analogue thereof, including carbetocin, stimulates myoepithelial contraction of the alveolar-ductal tissue which results in expression of intraductal fluid from a nipple of an individual. In some embodiments, the oxytocin, or an analogue thereof, including carbetocin, is administered intranasally. In some embodiments, the oxytocin, or an analogue thereof, including carbetocin. is administered by intramuscular or intravascular injection. In some embodiments, oxytocin, or an analogue thereof, including carbetocin, administered in an amount that is effective to stimulate expression of intraductal fluid from the nipple.

Once a sufficient post-administration time period has elapsed to allow the oxytocin to reach and stimulate target alveolar-ductal tissues, intraductal fluid is collected directly from the nipple. After the intraductal fluid is collected a bioassay is conducted on the intraductal fluid to determine the presence and/or amount of biomarkers of a breast disorder.

Any suitable oxytocin, or analogue thereof, including carbetocin. preparation is used for the methods disclosed herein

### Preparation of the Breast

Disclosed herein, in certain embodiments, are methods of diagnosing or prognosing a breast disorder in an individual in need thereof comprising screening a sample of intraductal fluid obtained during mammography of a breast of the individual for markers of a breast disorder and/or. markers associated with better clinical outcome in specific breast disorders such as T-cell markers. In some embodiments, the methods further comprise cleaning a nipple of the breast.

The nipple is cleaned by any suitable method. In some embodiments, the nipple is sterilized. In some embodiments, debris (e.g., keratin plugs) is removed from the nipple, increasing access to ducts of the nipple. In some embodiments, the nipple is scrubbed with a mild scrub with a dekeratinizing gel. In some embodiments, the nipple is scrubbed with an exfoliant. Any suitable exfoliant may be used with the methods disclosed herein. Examples of suitable exfoliants include, but are not limited to, microfiber cloths, adhesive exfoliation sheets, micro-bead facial scrubs, crepe paper, crushed apricot kernel or almond shells, sugar or salt crystals, pumice, and abrasive materials such as sponges, loofahs, brushes, salicylic acid, glycolic acid, fruit enzymes, citric acid, malic acid, alpha hydroxy acids (AHAs), and beta hydroxy acids (BHAs). In some embodiments, cleaning the nipple results in the opening of ducts of the nipple. In some embodiments, the ducts of a nipple are about 0.1 to about 0.3 mm in diameter after cleaning.

The ducts of the nipple have circular smooth muscles which tend to keep the lumen of the duct closed. In some embodiments, atropine or a related muscle relaxant such as tropicamide or phenylephrine is administered to the individual.

### Processing of Intraductal Fluid

Disclosed herein, in certain embodiments, are methods of diagnosing or prognosing a breast disorder in an individual in need thereof comprising screening a sample of intraductal fluid obtained during mammography of a breast of the individual for markers of a breast disorder. In some instances, the solid phase sample collection medium is washed following collection of the intraductal fluid obtained during mammography.

In some embodiments, washing the solid phase collection medium removes adherent interfering materials. In some embodiments, washing the solid phase collection medium comprises contacting it with a buffer solution, a detergent, or water. Exemplary detergents include, but are not limited to, Tween 20 (polyoxyethylene sorbitan monolaurate, Tween 80 (polyoxyethylene sorbitan mono-oleate), Triton X-100 (octyl phenoxy polyepoxy ethanol), TRIzol® (Life Techonologies, CA), and myristyltrimethyl ammonium bromide. In some embodiments, effluent of such washings is analyzed using methods including, but not limited to, microscopy, immunocytochemistry and flow cytometry. For example, after washing the absorbent paper or membrane containing the intraductal fluid sample to remove any cells, the wash solution is assessed using microscopy and the number of cells in the effluent is determined. In some embodiments, morphology of any cells present in the effluent is determined. In some embodiments, cell present in the effluent are stained for one or more extracellular and/or intracellular markers to determine if the cells have a normal profile or have one or more markers indicative of cancer cells. For example, cells may be analyzed for the presence or absence of BRCA1, BRCA2, p63, a cyclin, a cytokeratin, Her2 or any other marker that may indicate that the cells are cancer cells or normal cells based on the presence, absence, or level of such markers.

In other embodiments, after washing the absorbent paper or membrane containing the intraductal fluid sample, the wash solution is assessed for the presence of proteins, DNA such as cfDNA and mitochondrial DNA, and RNA such as cell-free RNA and microRNA (including pri-miRNA, pre-miRNA, and mature miRNA). The DNA or RNA is extracted and then subjected to further analyses as disclosed herein. Examples of analyses that may be performed include, without limitation, determination of DNA copy number changes, chromosomal aberrations, DNA mutations (duplications, deletions, inversions etc.) and single nucleotide polymorphisms (SNPs), DNA methylation, histone methylation, and protein methylation miRNA expressions or signatures, changes in lectin signatures, and changes in micro-biome.

In yet other embodiments, after washing the solid phase collection medium such as a absorbent paper or membrane containing the intraductal fluid sample, the solid phase collection medium is subjected to further analyses as disclosed herein.

In some embodiments, prior to, or concurrent with, an assay a preliminary evaluation is performed to verify sample origin and/or quality of the sample of intraductal fluid obtained during mammography. The focus of such preliminary evaluations is to verify that the sample collected from intraductal fluid obtained during mammography is indeed of mammary origin, and is not contaminated with other potential contaminants, such as sweat from skin surrounding the nipple. Other mammary fluid markers for sample verification include, but are not limited to, cytokeratins that are characteristically expressed by normal and cancerous mammary epithelial cells, and human mammary epithelial antigens (HME-Ags) corresponding to glycoprotein components of the human milk fat globulin (HMFG) protein.

### Screening and Classification

Disclosed herein, in certain embodiments, are methods of diagnosing or prognosing a breast disorder in an individual in need thereof comprising screening a sample of intraductal fluid obtained during mammography of a breast of the individual for markers of a breast disorder. In some embodiments, the methods disclosed herein are useful as companion diagnostic methods used as adjunct to mammography, particularly in individuals with BI-RADS categories II-IV. Accordingly, disclosed herein in some preferred embodiments, are methods of diagnosing or prognosing a breast disorder in an individual having a BI-RADS III or BI-RADS IV lesion, comprising screening intraductal fluid obtained from a nipple of the individual during mammography for at least one biomarker associated with a breast disorder.

In other embodiments, the methods are useful in predicting treatment selection, therapeutic outcome such as patient relapse and disease recurrence, survival and response to therapy such as chemotherapy, hormonal therapy, radiation therapy, etc. In yet other embodiments, the methods disclosed herein are useful in monitoring an individual's response to therapeutic treatment.

Breast disorder markers that can be determined in intraductal fluid sample comprises cytology, gene mutations (including substitutions, deletions, and inversions), single nucleotide polymorphisms (SNPs), DNA copy number, DNA methylation patterns or signatures, histone methylation patterns or signatures, microRNA patterns, micro-biome pattern, other disease biomarkers or a combination thereof. For a general and non-exhaustive review of breast disorder markers that are useful for the purpose of the present invention, see Hirata et al. Disease Markers, 2014, vol 2014, article ID 513158; Pultz, et al. J. Cancer. 2014, vol 5, pages 559-571; Lari and Keurer, J. Cancer. 2011, vol 2, pages 232-261, all of which are incorporated herein in their entirety.

In some embodiments, the screening comprises cytology, immunohistochemistry, immunocytochemistry, FISH, ICH, RIA, or any combinations thereof. In a preferred embodiment, the screening comprises ELISA. In other embodiments, the screening comprises amplification, sequencing, restriction fragment length polymorphism, and microarray or multiplex analyses of genes, polynucleotides, DNA (including cfDNA and mitochondrial DNA), RNA (including mRNA, cell free or exosomal miRNA, and mitochondrial RNA), fatty acids and glycoproteins, and lectins.

In some embodiments, the amplification is performed by ligase chain reaction (LCR) or polymerase chain reaction (PCR) including, without limitation, reverse-transcriptase (RT-PCR), quantitative PCR (qPCR), quantitative RT-PCR (qRT-PCR), real-time PCR, isothermal PCR, multiplex PCR, methylation-specific PCR, and the like.

In other embodiments, sequencing is dideoxy sequencing, reverse-termination sequencing, next generation sequencing, barcode sequencing, paired-end sequencing, pyrosequencing, multiplex sequencing, sequencing-by-synthesis, sequencing-by-hybridization, sequencing-by-ligation, single-molecule sequencing, single molecule real-time sequencing-by-synthesis, bisulfite-sequencing, whole genome sequencing, and whole exome sequencing. In yet other embodiments, the sequencing is by RNA-seq, Whole Transcriptome Shotgun Sequencing, and mRNA-Seq. In some embodiments where the breast disorder is associated with microbial dysbiosis, 16S RNA sequencing is preferred. In some preferred embodiments, sequencing is deep sequencing or ultra-deep sequencing.

One of skill in the art will recognize that it is within the scope of the present invention that screening comprises any combination of the methods disclosed herein and includes other methods known in the art.

In some preferred embodiments, screening is performed using a single cell, a plurality of cells, single nucleus and a plurality of nuclei of the intraductal fluid sample. In other preferred embodiments, the screening is performed on cell-free intraductal fluid sample.

In some embodiments, screening for or assessing DNA methylation is performed using any one or more of bisfulfite sequencing, methylation sensitive PCR, methylated DNA immunoprecipitation (MeDIP), methyl sensitive single nucleotide primer extensions (MS-SNuPE), genome-wide methylation profiling, methylation sensitive restriction enzyme analysis, combined bisulfite restriction analysis, methylation-specific quantum dot fluorescence resonance energy transfer (MS-qFRET), whole genome mapping or a combination thereof. In some embodiments, the DNA methylation screening further includes microarray or multiplex hybridization, gene expression, copy number analysis, next-generation sequencing, or a combination thereof.

Breast ducts contain two types of epithelial cells, inner luminal cells and outer basal/myoepithelial cells. In some embodiments, biomarker expression (e.g., by immunohistochemical staining, by differential gene expression, miRNA pattern/signature, and DNA methylation pattern/signature, histone methylation pattern/signature, etc.), genotyping, determining the absence or presence of genetic markers, including gene mutations (deletions, insertions, duplications and inversions), SNPs, etc. is used to distinguish between luminal and basal breast cancers, between hyperplasia, precancers, non-invasive and invasive cancers and metastasized cancers. In some preferred embodiments, biomarker expression (e.g., by immunohistochemical staining, alterations in miRNA pattern/signature, and DNA and/or histone methylation pattern/signature) is used to distinguish between hyperplasia of the usual type and atypical hyperplasia, between CCLs and hyperplasia, between precancers and cancers, between invasive and non-invasive cancers, etc. Tumor protein p63 (or, transformation-related protein 63) is a member of the p53 family of nuclear transcription factors. The presence of p63 characterizes the basal epithelial layer. In some embodiments, the presence of p63 in an intraductal fluid sample indicates that a breast cancer is basal-like breast cancer.

The presence of cytokeratin (CK) 5 and CK14 characterizes the basal epithelial layer. In some embodiments, the presence of CK5 and CK14 in an intraductal fluid sample indicates that a breast cancer is basal-like breast cancer. Further, the presence of CK5 and CK14 characterizes progenitor and myoepithelial cells. In some embodiments, the presence of CK5 and CK14 in an intraductal fluid sample indicates that the cell is a myoepithelial cell or a progenitor cell.

The presence of CK7 and CK18 characterizes the luminal epithelial layer. In some embodiments, the presence of CK7 and CK18 in an intraductal fluid sample indicates that a breast cancer is luminal breast cancer.

Usual ductal hyperplasia displays a luminal staining pattern with expression of both CK5/14 and CK7/18. Residual p63 is observed in the nuclei of the myoepithelium. In some embodiments, the presence of CK5, CK14, CK7, and CK18 indicates that a hyperplasia is usual ductal hyperplasia.

Atypical ductal hyperplasia or ductal carcinoma in situ display the differentiated glandular immunophenotype (CK7/CK18 positive), but are CK5/14-negative except for the myoepithelium. In some embodiments, the presence of CK7/CK18 and the absence of CK5/14 indicate that a hyperplasia is atypical ductal hyperplasia.

Invasive breast lesions are identified by a reduction in the number of or absence of myoepithelial cells (CK5/14 and/or p63) and the presence of glandular epithelial cells (CK7/18). In some embodiments, the presence of reduced or under-stress myoepithelial cells in the context of a suspected breast cancer indicates a transition to infiltrating and possibly invasive status. Primary breast carcinomas show an increase in the number of luminal (duct-wall) cells and a decrease in the number of myoepithelial cells. As a breast cancer evolves from in-situ, to infiltrating, and finally to invasive, the relative number of myoepithelial cells decreases. If the finding is for larger than normal numbers of luminal cells, it suggests that myoepithelial cells are diminishing in number, and there is cause for concern. In some embodiments, the absence of or a reduction in the number of myoepithelial cells and the presence of glandular epithelial cells indicates that the lesion is invasive.

In some embodiments, biomarker expression is determined by immunohistochemistry. In some embodiments, the immunohistochemistry method is a direct method. In some embodiments, a cell isolated from a sample of intraductal fluid obtained in conjunction with mammography is contacted with a labeled antibody which binds to a target antigen (e.g., p63, CK5, CK7, CK14, CK18, ER, PR, Her-2, Ki67, uPA, PAI-1, and galactose-N-acetylgalactosamine (Gal-GalNAc)). Any suitable label is used with a method disclosed herein. In some embodiments, the label is a dye (or, stain). In some embodiments, a different dye is used for each antibody. In some embodiments, the same dye is used for antibodies that bind to biomarkers present in the same cells type. For example, a first dye is used for antibodies that bind to biomarkers present in luminal breast cancer cells (CK7/18) and a second dye is used for antibodies that bind to biomarkers present in basal breast cancer cells (CK5/14 and p63).

In some embodiments, the immunohistochemistry method is an indirect method. In some embodiments, a cell isolated from an intraductal fluid sample is contacted with an unlabeled primary antibody and binds to the target antigen (e.g., p63, CK5, CK7, CK14, CK18) and a labeled secondary antibody binds to the primary antibody. In some embodiments, the primary antibody binds to a biomarker (e.g., CK5, CK7, CK14, CK18, or p63). In some embodiments, horseradish peroxidase (HRP) secondary antibodies bind to antibodies that bind to CK5/14 and p63. In some embodiments, alkaline phosphatase (AP) secondary antibodies bind to antibodies that bind to CK7/18. In some embodiments, a secondary antibody is raised to react with a primary antibody based on the species origin of the primary antibody, e.g., if the primary antibody is a mouse antibody then the secondary antibody would be, for example, a rabbit anti-mouse antibody. In a preferred embodiment, a conjugated goat anti-mouse poly-alkaline phosphatase (ALP) and a conjugated goat anti-rabbit poly-horseradish peroxidase (HRP) are used as secondary antibodies and react with both heavy and light chains on mouse and rabbit IgG.

In some embodiments, a chromogen (e.g., 3,3′ diaminobenzidine (DAB)) binds to the HRP and produces a chromogenic reaction product. Where the chromogen is DAB, the chromogen reaction product is brown. When the chromogen is Bajoran Purple, the chromogen reaction product is lavender-purple.

In some embodiments, a chromogen (e.g., Fast Red (FR)) binds to the AP and produces a chromogenic reaction product. Where the chromogen is FR, the chromogen reaction product is red or pink. In some embodiments, a cell isolated from a intraductal fluid sample is contacted with a peroxide block before contact with the primary antibody. Where the chromogen is Ferangi Blue, the chromogen reaction product is a bright royal blue.

In some embodiments, the cells are counterstained. In some embodiments, the cells are counterstained with hematoxylin, Nuclear Fast Red, Methyl Green, or Methyl Blue.

Disclosed herein, in certain embodiments, are methods of classifying a breast cancer as basal-like, comprising: (a) contacting a plurality of cells in a sample of intraductal fluid obtained during mammography with antibodies that bind to CK5, CK14, CK7, and CK18; and (b) classifying the cancer as basal-like if the CK5 and CK14 antibodies bind to the cells. In some embodiments, a method of classifying a breast cancer as basal-like comprises: (a) contacting a plurality of cells in a sample of intraductal fluid obtained during mammography with antibodies that bind to CK5, CK14, CK7, CK18, and p63; and (b) classifying the cancer as basal-like if the CK5, CK14, and p63 antibodies bind to the plurality of cells in the sample of intraductal fluid obtained during mammography.

Disclosed herein, in certain embodiments, are methods of classifying a breast cancer as luminal, comprising: (a) contacting a plurality of cells in a sample of intraductal fluid obtained during mammography with primary antibodies that bind to CK5, CK14, CK7, CK18, and p63; and (b) classifying the cancer as luminal if (i) the anti-CK7 and anti-CK18 primary antibodies bind to the plurality of cells, and (ii) the anti-CK5, anti-CK14, and anti-p63 primary antibodies do not bind to the plurality of cells in the sample of intraductal fluid obtained during mammography.

Disclosed herein, in certain embodiments, are methods of classifying a hyperplasia usual ductal hyperplasia, comprising: (a) contacting a plurality of cells in a sample of intraductal fluid obtained during mammography with primary antibodies that bind to CK5, CK14, CK7, CK18, and p63; and (b) classifying the hyperplasia as an usual ductal hyperplasia if the CK5, CK14, CK7, CK18, and p63 primary antibodies bind to the plurality of cells in the sample of intraductal fluid obtained during mammography.

Disclosed herein, in certain embodiments, are methods of classifying a hyperplasia as atypical ductal hyperplasia, comprising: (a) contacting a plurality of cells in a sample of intraductal fluid obtained during mammography with primary antibodies that bind to CK5, CK14, CK7, CK18, and p63; and (b) classifying the hyperplasia as atypical ductal hyperplasia if (i) the CK7 and CK18 primary antibodies bind to the plurality of cells in the sample of intraductal fluid obtained during mammography. In some embodiments, are methods of classifying a hyperplasia as atypical ductal hyperplasia, comprising: (a) contacting a plurality of cells in a sample of intraductal fluid obtained during mammography with primary antibodies that bind to CK5, CK14, CK7, CK18, and p63; and (b) classifying the hyperplasia as atypical ductal hyperplasia if CK5, CK15 and p63 bind to the plurality of cells in the sample of intraductal fluid obtained during mammography.

Disclosed herein, in certain embodiments, are methods of classifying a breast cancer as invasive, comprising: (a) contacting a plurality of cells in a sample of intraductal fluid obtained during mammography with primary antibodies that bind to CK5, CK14, CK7, CK18, and p63; and (b) classifying the cancer as invasive if the ratio of cells binding the CK5, CK14, and p63 primary antibodies to cells binding the CK7 and CK18 primary antibodies is less than or equal to an invasive control. In some embodiments, no cells in the sample of intraductal fluid bind CK5, CK14, and p63.

Disclosed herein, in certain embodiments, are methods of classifying a breast cancer as non-invasive, comprising: (a) contacting a plurality of cells in a sample of intraductal fluid obtained during mammography with primary antibodies that bind to CK5, CK14, CK7, CK18, and p63; and (b) classifying the cancer as non-invasive if the ratio of cells binding the CK5, CK14, and p63 primary antibodies to cells binding the CK7 and CK18 primary antibodies is greater than or equal to a non-invasive control.

Disclosed herein, are methods of detecting a breast disorder, comprising contacting a cell from the intraductal fluid sample collected from an individual during mammography with antibodies that bind to uPA, PAI-1, and Gal-GalNAc. In some preferred embodiments, the methods of detecting a breast disorder comprises contacting a cell from the intraductal fluid sample collected during mammography from an individual having BI-RADS II, BI-RADS III or BI-RADS IV lesions with antibodies that bind to uPA, PAI-1, and Gal-GalNAc.

In some embodiments, the methods comprise contacting the intraductal fluid sample with an antibody to any one or more of tryptophan degrading enzymes such as indoleamine 2, 3-dioxygenase-1 (IDO-1), indoleamine 2, 3-dioxygenase-2 (IDO-2), tyrosine 2.3-dioxygenase (TDO) or a combination thereof. IDO-1. IDO-2 and TDO activity are implicated in regulating T-cell activity via Tregs, regulating immune suppression in cancer subjects, and conferring tumoral immune resistance IDO-1, IDO-2 and TDO are said to aid in tumor escape. Thus, methods disclosed here are useful for classifying a breast cancer as invasive, comprising: (a) contacting one or more cells in a sample of intraductal fluid obtained during mammography with primary antibodies that bind to IDO-1. IDO-2, TDO or a combination thereof; and (b) classifying the cancer as invasive if the ratio of cells binding to IDO-1, IDO-2 TDO or a combination thereof is greater than or equal to a non-invasive control. In some preferred embodiments, the methods of detecting a breast disorder comprises contacting a cell from the intraductal fluid sample collected during mammography from an individual having BI-RADS II, BI-RADS III or BI-RADS IV lesions with antibodies that bind to IDO-1, IDO-2 TDO or a combination thereof.

In some embodiments, following collection of the intraductal fluid sample, the absorbent paper is washed and the effluent is collected and assessed for number of cells. Where the sample is acellular, in some embodiments the patient is identified as at low risk for breast cancer. Where the sample comprises one cell, in some embodiments the patient is identified as at low risk for breast cancer, and optionally the cells are assayed for biomarker expression. Where the sample comprises 2 or more cells, in some embodiments the patient is identified as at risk for breast cancer and the cells are assayed for biomarker expression.

In some embodiments, cytology of cells, if any, in said sample is analyzed using any suitable methods including, but not limited to, microscopy, flow cytometry, immunohistochemistry, or a combination thereof. In one non-limiting example, cell samples may be stained with hemolysin and eosin.

In some embodiments, protein measured by the methods described herein is total protein content of the sample. In some embodiments, the absorbent paper is exposed to colloidal gold or colloidal silver and total protein content (concentration) is determined using any suitable methods. In some embodiments, the absorbent paper is pre-loaded or pre-coated with colloidal gold or colloidal silver before contacting a breast of an individual. The term “colloidal metal particles” used in this connection is meant to include dispersions of particles, preferably sols, consisting of a metal, a metal compound or nuclei coated with a metal or metal compound. The terms “gold colloid” and “colloidal gold composition” used herein refer to a suspension of sub-micrometer-sized gold particles evenly dispersed in a fluid (e.g., water or an aqueous buffer). The colloidal gold composition utilized in the quantification assay contains highly concentrated gold particles. In one example, the colloidal gold composition has a gold particle concentration ranging from 3.5×10¹² to 7.0×10¹² particles/ml, e.g., (3.5-5.25)×10¹² particles/ml.

In some embodiments, total protein concentration of the sample of intraductal fluid obtained during mammography is determined and, if the total protein concentration of the sample is greater than 300 ng, the patient is identified for further assessment of breast cancer. In some embodiments, the total protein concentration of the sample of intraductal fluid obtained during mammography is determined and, if the total protein concentration of the sample is at, or below 200 ng protein, the patient is identified as being at low risk for breast cancer. In some embodiments, the total protein concentration of the sample of intraductal fluid obtained during mammography is determined and, if the total protein concentration of the sample is from about 300 ng to about 2 ug, the patient is identified as being at an elevated risk for breast cancer.

In some embodiments, if the sample of intraductal fluid obtained during mammography comprises from about 50 pg to about 0.5 ng of protein, and does not contain cells, a patient is identified as being at low risk for breast cancer.

In some embodiments, if the sample of intraductal fluid obtained during mammography contains at least about 300 ng of protein and two (2) or more cells, a patient is identified for further evaluation of breast cancer or diagnosed as at medium or high risk for breast cancer. In some embodiments, the cell fraction of the sample of intraductal fluid obtained during mammography, comprises between about 2 cells to about 50 cells. In some embodiments, the cell fraction of the sample of intraductal fluid obtained during mammography comprises at least ten (10) cells.

In some embodiments, the intraductal fluid sample comprises miRNA. In some embodiments, miRNA are cell free or exosomal. In some embodiments, screening the intraductal fluid sample comprises determining the presence and/or the level of one or more miRNA, profiling miRNA signature, or a combination thereof in the intraductal fluid sample. In some embodiments, miRNA comprise oncomirs, tumor suppressor miRNA, or a combination thereof. One of skill in the art will recognize that a miRNA pattern or signature may include both oncomirs and tumor suppressor miRNAs and that this signature may be altered in an individual with a breast disorder compared to an individual without a breast disorder. In some embodiment, the miRNA signature may be altered in one or both breasts of an individual with a breast disorder. In certain embodiments, intraductal fluid samples are screened to stratify or classify individuals comprising at least one oligonucleotide probe or primer capable of binding to at least a portion of a miRNA in intraductal fluid sample. In other embodiments, the intraductal fluid samples are screened to stratify or classify individuals comprising a plurality of oligonucleotide probes or primers capable of binding to at least a portion of miRNA in the intraductal fluid sample. In other embodiments, intraductal fluid samples are screened to further classify individuals having a BI-RADS III or a BI-RADS IV lesion.

Disclosed herein, in some embodiments, are methods for classifying a individual as having a breast disorder comprising: a) obtaining intraductal fluid sample from a nipple of the individual during mammography, and b) detecting the presence of one or more miRNA selected from Table 3, Table 4, Table 5 or a combination thereof in the intraductal fluid, wherein the presence of the miRNA is detected when the miRNA has a measured value above a threshold value for the miRNA; and c) classifying the individual having a breast disorder if the detected miRNA has a measured value above said threshold value.

Disclosed herein, in some embodiments, are methods for classifying a individual as having a breast disorder comprising: a) obtaining intraductal fluid sample from a nipple of the individual undergoing mammography, and b) detecting the decreased presence of one or more miRNA selected from Table 3, table 4, table 5 or a combination thereof in the intraductal fluid, wherein the decreased presence of the miRNA is detected when the miRNA has a measured value below a threshold value for the miRNA; and c) classifying the individual having a breast disorder if the detected miRNA has a measured value below said threshold value.

TABLE 3
Altered miRNA in Breast Disorders

| Columnar Cell Hyperplasia | Expression |
|---|---|
| Epithelial cells | |
| Let-7c | Down |
| miR-27a | Down |
| miR-92a | Down |
| miR-383 | Down |
| miR-202 | Down |
| miR-107 | Down |
| miR-141 | Down |
| miR-183 | Up |
| miR-454 | Up |
| Columnar Cell Hyperplasia | Expression |
| Stromal Cells | |
| miR-650 | Down |
| miR-335 | Down |
| miR-566 | Down |
| miR-497 | Down |
| miR-27a | Down |
| miR-204 | Down |
| miR-20a | Down |
| miR-132 | Up |
| miR-539 | Up |
| miR-221 | Up |
| Atypical Ductal Hyperplasia | Expression |
| miR-21 | Up |
| miR-183 | Up |
| miR-200c | Up |
| miR-200b | Up |
| miR-638 | Down |
| miR-572 | Down |
| miR-671-5p | Down |
| miR-30d | Up |
| miR-1275 | Down |
| miR-15b | Up |
| miR-644 | Up |
| miR-141 | Up |
| DCIS | |
| miR-195 | Down |
| miR-557 | Down |
| miR-1207-5p | Down |
| miR-874 | Down |
| miR-556-3p | Up |
| IDC | |
| miR-933 | Down |
| miR-141 | Up |
| miR-96 | Up? |
| miR638 | Down |
| miR-575 | Down |
| Let-7f | Up |
| miR-15a | Up |
| miR-671-5p | Down |
| miR-20a | Up |
| miR-1202 | Down |
| miR-183 | Up |
| miR-143 | Up |
| miR-19b | Up |
| miR-1915 | Down |
| miR-107 | Up |
| miR-21 | Up |
| miR-1274b | Up |
| miR-1268 | Down |
| miR-200b | Up |
| miR-106b | Up |
| miR-634 | Down |
| miR-129 | Down |
| miR-572 | Down |
| miR-933 | Down |
| miR-17 | Up |
| miR-29b | Up |
| miR-877 | Up |
| miR-425 | Up |
| miR-181a | Down |
| miR-193a | Down |
| miR-193b | Down |
| miR-145 | Down |
| miR-17-5p | Down |
| miR-20a | Down |
| miR-30b | Up |
| miR-30d | Up |

TABLE 4
Tumor Suppressor miRNA in Breast Cancers

| Cell Growth/Proliferation | Invasion/metastasis |
|---|---|
| miR-34a | miR-340 |
| miR-17-5p | miR-34a |
| miR-125b | miR-145 |
| miR-146a | miR-183 |
| miR-128 | miR-17 |
| | miR-20 |
| Cell Survival | miR-26a |
| miR34a | |
| Immune recognition | Angiogenesis |
| miR-322 | miR-145 |
| miR-93 | miR519c |
| miR-181a | miR340 |

In some embodiments, the presence of a miRNA is detected using any of the PCR methods known in the art. Such PCR methods include, without limitation, RT-PCR, real-time PCR, semi-quantitative PCR, qPCR, multiplex PCR or isothermal PCR. In other embodiments, a miRNA may be detected by hybridization to one or more miRNA probes which may be comprised on a microarray or a biochip or in a hybridization solution. In some preferred embodiments, a miRNA signature may be determined by miRNA microarray or multiplex hybridization and analysis. In some embodiments, the one or more miRNA probes may be attached to a solid phase sample collection medium (such as in a multiplex or on a microarray). In some embodiments, the miRNA probe(s) may be attached to a solid phase sample collection medium made of a material such as glass, modified or functionalized glass, plastics, nylon, cellulose, or nitrocellulose papers, resins, silica or silica-based materials etc. The miRNA probes may be attached to the solid phase sample collection medium covalently or non-covalently.

The diagnosis or prognosis may be based on differential expression of the miRNA in the intraductal fluid samples from normal subjects compared to samples from subjects with breast disorders.

In some embodiments, the methods for classifying an individual as at risk for or having CCH, ADH, DCIS or IDC comprising: a) obtaining intraductal fluid from a nipple of the individual during mammography, and b) detecting an altered expression of at least one miRNA selected from Table 3.

In some embodiments, the methods for classifying an individual as having Columnar Cell Hyperplasia comprises: a) obtaining intraductal fluid from a nipple of the individual during mammography, and b) detecting altered expression of miRNA selected from Table 3. Preferred embodiments for classifying an individual as having CCH include screening for at least one or more miRNA selected from the group consisting of Let-7c, miR-27a, miR-92a, miR-383, miR-202, miR-107, miR-141, miR-183, miR-454, miR-650, miR-335, miR-566, miR497, miR-27a, miR-204, miR-20a, miR-132, miR-539, and miR-221 in the intraductal fluid sample of an individual having a BI-RADS II, BI-RADS III or BI-RADS IV lesion obtained from a nipple of the individual during mammography. Altered expression of Let-7c, miR-27a, miR-92a, miR-383, miR-202, miR-107, miR-141, miR-183, and/or miR-454 would be indicative of CCH of epithelial cells whereas altered expression of miR-650, miR-335, miR-566, miR497, miR-27a, miR-204, miR-20a, miR-132, miR-539, and/or miR-221 would be indicative of CCH of stromal origin.

In some embodiments, the methods for classifying an individual as having Atypical Ductal Hyperplasia comprises: a) obtaining intraductal fluid from a nipple of the individual during mammography, and b) detecting the altered expression of at least one miRNA selected from Table 3. Preferred embodiments for classifying an individual as having ADH include screening for at least one or more miRNA selected from the group consisting of miR-21, miR-183, miR-200c, miR-200b, miR-638, miR-572, miR-671-5p, miR-30d, miR-1275, miR-15b and miR-644 in the intraductal fluid sample of an individual having a BI-RADS III or a BI-RADS IV lesion obtained from a nipple of the individual during mammography.

In some embodiments, the methods for classifying an individual as having DCIS comprises: a) obtaining intraductal fluid from a nipple of the individual during mammography, and b) detecting the altered expression of at least one miRNA selected from Table 3. Preferred embodiments for classifying an individual as having DCIS include screening for at least one or more miRNA selected from the group consisting of miR-195, miR-557, miR-554, miR-1207-5p, miR-874, miR-556-3p, and miR-556-3p in the intraductal fluid sample of an individual having a BI-RADS III or a BI-RADS IV lesion obtained from a nipple of the individual during mammography.

In some embodiments, the methods for classifying an individual as having IDC comprises: a) obtaining intraductal fluid from a nipple of the individual during mammography, and b) detecting the altered expression of at least one miRNA selected from Table 3. Preferred embodiments for classifying an individual as having IDC include screening for at least one or more miRNA selected from the group consisting of miR-933, miR-141, miR-96, miR638, miR-575, Let-7f, miR-15a, miR-671-5p, miR-20a, miR-1202, miR-183, miR-141, miR-19b, miR-1915, miR-107, miR-21, miR-1274b, miR-1268, miR-200b, miR-106b, miR-634, miR-129, miR-572, miR-933, miR-17, miR-29b, miR-877, miR-425, miR-23b, miR-193a, miR-193b, miR-181a, miR-143, miR-145, miR-17-5p, miR-20a, miR-30b, and miR-30d in the intraductal fluid sample of an individual having a BI-RADS III or a BI-RADS IV lesion obtained from a nipple of the individual during mammography.

Targets of tumor suppressor miRNAs disclosed in Table 4 are implicated in breast cancer and are known in the art (Modulation of Cancer Traits by tumor Suppressor microRNAs. Grammatikakis, I. et al. Int. J. Mol. Sci. 2013, vol 14, pages 1822-1842; microRNA 17/20 inhibits cellular invasion and tumor metastasis in breast cancer by heterotypic signaling. Yu, et al. Proc. Natl. Acad. Sciences. 2010, vol. 107(18), pages 8231-8236, each incorporated in its entirety herein). One of skill in the art will recognize that such targets fall within the scope of the present invention and that the methods disclosed herein include the targets as biomarkers of breast disorder.

TABLE 5
miRNA associated with altered target expression
in invasive breast cancers

| | | GalNAc | |
|---|---|---|---|
| uPA | PAI-1 | transferases | IDO-1 |
| miR-23b (down) | miR-143 | miR-30b (Up) | miR-181a (down) |
| miR-193a (down) | miR-145 | miR-30d (Up) | |
| miR-193b (down) | miR-17-5p (down) | miR-548a-3p | |
| miR-181a (down) | miR-20a (down) | miR-183* | |
| | | miR-124 | |
| | | miR-29a* | |
| | | miR-506 | |
| | | miR-3143 | |
| | | miR-4324 | |
| | | miR-569 | |
| | | miR-548e | |
| | | miR-491-3p | |
| | | miR-3672 | |
| | | miR-544b | |
| | | miR-135b | |
| | | miR-2117 | |
| | | miR-590-3p | |
| | | miR-378* | |
| | | miR-135a | |

In some embodiments, the methods of classifying an individual undergoing mammography as at risk for or having breast cancer (CCH, ADH, DCIS, IDC, or LCIS) comprise obtaining intraductal fluid sample from a nipple of an individual during mammography and screening for altered expression of miRNA listed in Table 5 regulating uPA, PAI-1 and Gal-GalNAc expression in the individual. In some preferred embodiments, expression of miRNA listed in Table 5 regulating IDO1 expression are also screened. Preferred embodiments include screening for altered expression of miRNA selected from the group consisting of miR-23b, miR193a, miR193b, miR181a, miR143, miR145, miR-17-5p, miR-20a, miR30b, and miR-30d regulating uPA, PAI-1 and Gal-GalNAc transferases expression in the intraductal fluid sample of an individual having a BI-RADS III or a BI-RADS IV lesion obtained from a nipple of the individual during mammography. In some preferred embodiments, miRNA 181a regulating IDO-1 is also screened. In some embodiments, the methods for classifying a subject as having luminal-A like breast cancer comprises: a) obtaining intraductal fluid sample from a nipple of the subject undergoing mammography, and b) detecting the altered expression of at least one miRNA selected from the group consisting of miR-29a, miR181a, and miR-652.

In some embodiments, the methods of classifying a subject as at risk for or having invasive breast cancer comprises obtaining intraductal fluid sample from a subject undergoing mammography and screening for altered signature of miRNAs which regulate uPA, PAI-1, and GalNac Transferases. GalNac Transferases 3, 6 and 7 are preferred GalNAc Transferases. More preferred GalNac transferases are Beta 1→3 galactosyltransferases such as B3GALT1 and B3GALT5. Such methods comprise screening for altered miRNA signatures that upregulate uPA, PAI-1 and Gal-GalNac expression. In some embodiments, the methods comprise screening for altered signature of miRNA regulating IDO-1, IDO-2, TDO or a combination thereof. IDO1 and -2 and TDO are known to aid in tumor escape and are associated with cancer-associated immune suppression. In some embodiments, methods comprise screening for altered signature of miRNAs which regulating uPA, PAI-1, GalNac Transferases, and IDO-1. Accordingly, in some preferred embodiments, the methods for classifying a subject at risk for or having invasive breast cancer comprises: a) obtaining intraductal fluid sample from a nipple of the subject undergoing mammography, and b) detecting a altered levels of miR-23b, miR193a, miR-193b, miR-181a, miR-143, miR-145, miR-17-5p, miR-20a, miR-548a-3p, miR-183*, miR-124, miR-29a*, miR-506, miR-3143, miR-4324, miR-569, miR-548e, miR-491-3p, miR-3672, miR-544b, miR-135b, miR-2117, miR-590-3p, miR-378*, miR-135a, miR30b, and miR-30d. Decrease in levels of miR193a, miR-193b, and miR-181a and increase in the levels of miR30b and miR-30d would be indicative of increased risk of invasive breast cancer.

In some embodiments, increased presence or upregulation of miR-21, miR-494 and miR-183 would be indicative of increased risk and/or poor prognosis of cancer metastasis or cancer progression. In other embodiments, upregulation of let-7a, let-7b and let-7c and miR-1308 would be indicative of metastatic potential of the breast disorder. In some preferred embodiments, miR-200 family miRNA members such as miR-200b and miR-200c are the preferred diagnostics, prognostics and/or predictive metastatic disease markers.

Disclosed herein, in some embodiments are methods of classifying an individual as having a breast disorder, comprising: a) obtaining intraductal fluid sample from a nipple of the individual during mammography; b) assessing the individual's DNA methylation signature using the intraductal fluid sample; and c) classifying the individual as having breast disorder based on the individual's DNA methylation signature. In some embodiments, the individual is classified as having a beast disorder that is luminal A, luminal B, or basal-like breast cancer based on the DNA methylation signatures in the intraductal fluid sample. In other embodiments, based on the DNA methylation signatures of specific genes, an individual may be diagnosed or prognosed as having a risk for or having poor survival and/or relapse.

In some embodiments, the individual has DNA hypermethylation of at least one or more of genes listed in Table 6.

In some embodiments, the individual has DNA hypomethylation of at least one or more of genes susceptible to DNA methylation. For example, without limitation, hypomethylation or demethylation of genes such as uPA and PAI-1 would be indicative of increased risk of breast cancer.

In some preferred embodiments are methods of diagnosing or prognosing a breast disorder in an individual having a BI-RADS III or a BI-RADS IV lesion comprising: a) obtaining intraductal fluid sample from a nipple of the individual during mammography; and b) assessing the individual's DNA methylation signature using the intraductal fluid sample

Methods for genome wide and specific gene DNA methylation profiling useful for the purpose of this invention are known in the art, including a non-exhaustive list of such methods provided above. In some embodiments, screening for or assessing DNA methylation is performed by bisfulfite sequencing, methylation sensitive PCR, methylated DNA immunoprecipitation (MeDIP), methyl sensitive single nucleotide primer extensions (MS-SNuPE), genome-wide methylation profiling, methylation sensitive restriction enzyme analysis, combined bisulfite restriction analysis, methylation-specific quantum dot fluorescence resonance energy transfer (MS-qFRET), whole genome mapping or a combination thereof. In some embodiments, preferred assessment method is genome-wide DNA methylation profiling. DNA methylation profiling methods are known in the art (Dedeurwaerder, et al. EMBO Molecular Medicine, 2011). Commercial sources of such assays are available for e.g., Illumina Infinium Human Methylation27 Bead chip (Illumina)

TABLE 6
Target Genes for DNA methylation pattern/signature analyses

| | | | | | |
|---|---|---|---|---|---|
| ABCA3 | GSTP1 | UAP1L1 | RASSF1 | FZD9 | PTGS2 |
| COX7A1 | PTPRO | SYDE1 | B3GALT5 | B4GALT3 | B4GALT7 |
| SST | RECK | UGT3A2 | SCGB3A1 | FGFP3 | GAS7 |
| CDKL2 | ACADL | TNFRSF10D | HDAC9 | HOXA11 | MME |
| ZNF154 | SFRP2 | C1orF114 | POMC | RBP1 | BCR |
| APC | ITR | COL1A2 | C4B | DAB21P | MEST |
| CCND2 | UGT3A1 | SIT1 | RARA | SEPT5 | TFF1 |
| LAX1 | HCLS1 | CD3D | THY1 | SERPINA5 | ASCL2 |
| ICOS | CD6 | CD79B | DLK1 | EYA4 | HOXA5 |
| LCK | CCL5 | UBASH3A | HOXA9 | HOXB13 | IHH |
| CD3G | Gal-NAc | B3GALT1 | IPF1 | ISL1 | PAV6 |
| | trans- | | | | |
| | ferases | | | | |
| B3GALT5 | uPA | PAI-1 | TBX1 | SOX1 | SOX17 |
| MUC1 | P16 | FABP3 | CDH17 | EWPHX1 | B3GALT1 |
| APC | BIN1 | BRCA1 | B3GALT5 | B4GALT3 | B4GALT7 |
| BRCA2 | CST6 | GSTP1 | TIMP3 | P21 | PTEN |

Disclosed herein, in certain embodiments, are methods of classifying a individual as being a breast CpG island methylator phenotype (B-CIMP) comprising: a) obtaining intraductal fluid from a nipple of the individual during mammography; and b) characterizing the individual's CpG island methylation phenotype; wherein if the individual is characterized as B-CIMP, the individual is said to have a low risk of breast cancer metastasis and/or improved survival. Methods of characterizing an individual's phenotype as B-CIMP are known in the art (Fang, F et. Al, Sci Transl Med. 2011, 3:75ra25).

Disclosed herein, in some embodiments, are methods for classifying an individual as having a breast disorder comprising: a) obtaining an intraductal fluid sample from a nipple of the individual during mammography, and b) determining the individual's DNA methylation phenotype using the intraductal fluid sample; and c) characterizing the individual's DNA methylation signature as a breast CpG island methylator phenotype, wherein the individual is said to have a low risk of breast cancer metastasis and/or improved survival if the individual is classified having the breast CpG island methylator phenotype.

In some embodiments, are methods for classifying an individual as at risk for or having a luminal breast cancer comprising: a) obtaining an intraductal fluid sample from a nipple of the individual during mammography, and b) determining the DNA methylation status of one or more genes selected from the group consisting of RASSF1, FZD9, PTGS2, MME, HOXA9, PAX6, SCGB3A1, FABP3, FGFP3, GAS7, HDAC9, HOXA11, MME, PAX6, POMC, and RBP1, wherein the individual is characterized as at risk for or having luminal breast cancer if the genes are hypermethylated.

In some embodiments, are methods for classifying an individual as at risk for or having a basal-like breast cancer comprising: a) obtaining an intraductal fluid sample from a nipple of the individual during mammography, and b) determining the DNA methylation status of one or more genes selected from the group consisting of CDH17, EWPHX1, TH-1, RARA, MEST, BCR, C4B, SEPT5, SERPINA5, and THY1, wherein the individual is characterized as at risk for or having luminal breast cancer if the genes are hypermethylated.

In some embodiments, are methods for classifying an individual as at risk for or having a luminal breast cancer comprising: a) obtaining an intraductal fluid sample from a nipple of the individual during mammography, and b) determining the DNA methylation status uPA and PAI-1, wherein the individual is characterized as at risk for or having luminal breast cancer if the genes are hypomethylated.

Other preferred embodiments include assessing altered DNA methylation of ACADL, RECK and SFR2 in an individual having a BI-RADS III or a BI-RADS IV lesion using the intraductal fluid sample obtained from a nipple of the individual during mammography. An increased DNA methylation of one or more ACADL, RECK and SFR2 genes in an individual would be indicative of increased risk of relapse and/or poor survival. In some preferred embodiments include assessing altered DNA methylation of uPA and PAI-1 genes in an individual having a BI-RADS III or a BI-RADS IV lesion using the intraductal fluid sample obtained from a nipple of the individual during mammography. Hypomethylation of uPA and PAI-1 gene would be indicative of increased risk of breast cancer.

### Methods of Treatment

Disclosed herein, in certain embodiments, are methods of diagnosing or prognosing a breast disorder in an individual in need thereof comprising screening a sample of intraductal fluid obtained during mammography of a breast of the individual for markers of a breast disorder. In some embodiments, the method further comprises determining a treatment course for the subject based on results of the screening. In some embodiments, the method comprises determining a treatment course for an individual having a BI-RADS III or BI-RADS IV lesion. In some embodiments, a medical profession prescribes a treatment regimen to the individual based on the results of the screening. In some embodiments, the treatment regimen comprises a therapeutic agent, radiation therapy, and/or surgical excision of breast tissue. In some embodiments, the treatment regimen comprises a plurality of therapeutic agents.

In some embodiments, the therapeutic agent is an anthracycline (e.g., doxorubicin or epirubicin), a platinum agent, a taxane (e.g., paclitaxel or docetaxel), or a combination thereof. In some embodiments, the therapeutic agent is ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen. toremifene, trastuzumab, vinorelbine, or a combination thereof.

In some embodiments, the therapeutic agent is a SERM, a SERD, an AI, a pharmaceutical salt thereof, or a combination thereof. In some embodiments, the SERM is selected from the group consisting of tamoxifen, cis-tamoxifen, 4-hydroxytamoxifen (4-OHT), endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652, and ERA-92. Preferably, in some embodiments, the therapeutic agent is tamoxifen or a tamoxifen derivative (such as 4-hydroxytamoxifen, N-desmethyltamoxifen, endoxifen, and cis-tamoxifen). In some embodiments, the SERD comprises fulvestrant, ARN-810, or CH4986399. In some embodiments, the AI is selected from the group consisting of anastrozole, exemestane and letrozole. In some embodiments, the plurality of therapeutic agents comprise a SERD, a SERM, an AI, a pharmaceutical salt thereof, or a combination thereof. In some embodiments, the therapeutic agent comprises at least one omega-3 fatty acid and at least one vitamin D compound.

In some embodiments, the therapeutic agent is butyric acid. In some embodiments, the therapeutic agent is doxorubicin. In some embodiments, the therapeutic agent is epirubicin. In some embodiments, the therapeutic agent is paclitaxel. In some embodiments, the therapeutic agent is docetaxel.

In some embodiments, the therapeutic agent is a silencer of anti-tumor suppressor miRNA disclosed herein in Table 3, Table 4, Table 5, or a combination thereof. A non-limiting example of such anti-tumor suppressor miRNA is p63. In other embodiments, the therapeutic agent is an activator of an oncomir disclosed herein in Table 3, Table 4, Table 5, or a combination thereof. In some embodiments, the therapeutic agent is based on human or a non-human sequence of a miRNA disclosed herein. In some embodiments, an inhibitor of miRNA is an antisense oligonucleotide. Anti-sense oligonucleotide can include ribonucleotide or deoxyribonucleotides or a combination thereof. Antisense nucleotides may have one or more chemical modification, for example, sugar or backbone modification.

In some embodiments, the therapeutic agent is an inhibitor of DNA hypermethylation of genes that are hypermethylated in breast disorders as disclosed herein. One of skill in the art will recognize that inhibition of DNA hypermethylation will reduce gene silencing effect of certain genes implicated in breast disorders. In other embodiments, the therapeutic agent is an activator of DNA methylation or a DNA methylating agent. In such cases, the genes that are involved in cell proliferation and tumor formation will be silenced or have reduced expression.

In some embodiments, the therapeutic agent is a combination therapy. Where combination therapy is administered, each of the agents may be administered in combination with any other agent (e.g., simultaneously) or alone. Further, all of the agents may be administered according to the claimed method. Alternatively, some of the agents may be administered according to the claimed method, while others are administered systemically.

In some embodiments, the combination therapy is CAF: cyclophosphamide, doxorubicin, and 5-FU. In some embodiments, the combination therapy is TAC: docetaxel, doxorubicin, and cyclophosphamide. In some embodiments, the combination therapy is AC→T: doxorubicin and cyclophosphamide followed by paclitaxel or docetaxel. In some embodiments, the combination therapy is FEC:→T: 5-FU, epirubicin, and cyclophosphamide followed by docetaxel or paclitaxel. In some embodiments, the combination therapy is TC: docetaxel and cyclophosphamide. In some embodiments, the combination therapy is TCH: docetaxel, carboplatin, and trastuzumab for HER2/neu positive tumors. In some embodiments, the combination therapy is CMF: cyclophosphamide, methotrexate, and 5-fluorouracil. In some embodiments, the combination therapy is A→CMF: doxorubicin, followed by CMF. In some embodiments, the combination therapy is EC: epirubicin and cyclophosphamide. In some embodiments, the combination therapy is AC: doxorubicin and cyclophosphamide

### EXAMPLES

The present invention may be better understood through reference to the following examples. These examples are included to describe exemplary embodiments only and should not be interpreted to encompass the entire breadth of the invention.

### Example 1

### Assessment of an Intraductal Fluid Sample Obtained During Mammography

An absorbent paper is utilized as the solid phase collection medium. The individual is administered oxytocin before mammography. The nipples of both breasts are cleaned and keratin plugs removed. A nitrocellulose filter is attached to both nipples. Each breast is placed in a mammography device and mammography is performed.

Following collection of an intraductal fluid sample obtained during mammography, the nitrocellulose filter is washed using any suitable buffered wash solution (e.g., phosphate buffered saline). The effluent is collected in a modified cytology vial and centrifuged. Cells are isolated from the effluent and transferred to the central region of a clean glass microscopic slide, and a cover slip is applied. The slide is allowed to air dry and then is fixed, for example in absolute alcohol.

Monoclonal antibodies CK5, CK14, p63 and rabbit monoclonal antibodies CK7 and CK18 are multiplexed with a single antibody diluent and applied to the microscopy slide. A biotin-free multistain detection reagent composed of a cocktail of goat-anti-mouse-HRP and goat anti-rabbit-AP is then applied. DAB and Fast Red chromogens are applied sequentially. Cells are counterstained with hematoxylin.

| Antibody | Chromogen | Cell Type |
|---|---|---|
| CK5 | DAB | Progenitor cells |
| | Brown | Myoepithelial/luminal |
| | | Basal phenotype |
| CK14 | DAB | Progenitor cells |
| | Brown | Myoepithelial/luminal |
| | | Basal phenotype |
| P63 | DAB | Basal Myoepithelium |
| | Brown | Basal phenotype |
| CK7 | FR | Normal breast cells |
| | Red | Glandular epithelium |
| | | Luminal epithelium |
| CK18 | FR | Normal breast cells |
| | Red | Glandular epithelium |
| | | Luminal epithelium |

The results of the assays are compared with the mammogram x-ray results. If the results are consistent, no additional assays are performed. If the results are inconsistent, additional assays or tests are performed.

### Example 2

### Treatment of an Intraductal Fluid Sample Obtained During Mammography

An absorbent paper is utilized as the solid phase collection medium. The individual is administered oxytocin before mammography. The nipples of both breasts are cleaned and keratin plugs removed. A nitrocellulose filter is attached to both nipples. Each breast is placed in a mammography device and mammography is performed.

Following collection of an intraductal fluid sample obtained during mammography, the nitrocellulose filter is washed using any suitable buffered wash solution (e.g., phosphate buffered saline). The effluent is collected in a modified cytology vial and centrifuged. Cells are isolated from the effluent and transferred to the central region of a clean glass microscopic slide, and a cover slip is applied. The slide is allowed to air dry and then is fixed, for example in absolute alcohol.

### Pretreatment

The cells are contacted with a peroxide block—Biocare's Peroxidazed 1.

Next, perform heat retrieval pretreatment. Preheat Diva solution to 95° C. for 30 minutes in Biocare's Decloaking Chamber. Then, place slides into the preheated solution and retrieve under pressure at 95° C. for 40 minutes. Alternatively, steam tissue sections for 45-60 minutes or use a water bath at 95° C. for 40 minutes. Allow solution to cool for 20 minutes then wash in distilled water.

Apply protein block—Incubate for 10-15 minutes at RT with Biocare's Background Sniper.

Incubate the slide with the primary antibodies (i.e., antibodies to CK5, CK14, CK7, CK18, and p63) for 30-60 minutes at room temperature.

Incubate slide for 30 minutes at RT using Biocare's MACH 2 Double Stain 2.

Incubate for 5 minutes at RT when using Biocare's Betazoid DAB.

Incubate for 10-20 minutes at RT with Biocare's Vulcan Fast Red. Rinse in deionized water.

Rinse with deionized water. Incubate for 30-60 seconds with Hematoxylin. Rinse with deionized water. Apply Tacha's Bluing solution for 1 minute.

Visualize cells with a light microscope.

### Example 3

### Assessment of Intraductal Fluid

This trial was a single-center study involving three (3) healthy, non-pregnant, non-lactating female subjects. Subjects were enrolled in the order of appearance at the clinic.

The primary trial objective was to determine the percentage of women from age 30 to 65 that produces intraductal fluid during a mammogram procedure, as determined by the presence of protein on the nitrocellulose filter.

A secondary objective was to evaluate the intraductal fluid cytologically for the presence and type of cells (if any).

Methodology:

Briefly, a tared nitrocellulose filter was used to collect intraductal fluid expressed during mammography by adhering it to each nipple (one for each breast). In one set of subjects, a mammogram was performed. In a second set of subjects, a mammogram was not performed (control group). Cells collected from washing the filters containing intraductal fluid specimens underwent cytological examination.

Assessment:

The primary endpoint of the trial was the percentage of women completing the trial that produce intraductal fluid, as determined by the presence of protein on the nitrocellulose filter when undergoing mammography.

The secondary endpoint was the presence of cells in the intraductal fluid sample as determined by cytologic evaluation.

Results:

With regard to the protein testing done of the filters obtained from these subjects groups, none on the samples obtained from the control group showed the presence of protein. All filters from group that underwent a mammogram showed the presence of protein.

### Example 4

### Detection of miRNA in Intraductal Fluid Sample

Intraductal fluid sample will be aspirated from the nipples of both breasts of a female subject undergoing mammography using a collection device comprising a breast engaging member which attaches the device to the breast. The intraductal fluid sample will be collected onto a solid phase phase sample collection medium such as an absorbent paper. The intraductal fluid sample absorbed on the absorbent paper will be washed and the total RNA (including the miRNA) will be isolated as follows.

400 μL Trizol™ (Invitrogen®, Carlsbad, Calif.) will be added to the a 2 mL microfuge tube containing the absorbent papers of sizes 1 cm×1 cm and 1 inch×1 inch with the intraductal fluid sample. The tube will be vortexed at 2000 rpm for 30 m at 4° C. on a vortexer. Following further addition of 1.2 mL Trizol™ and then 0.24 mL chloroform, the tube will be vortexed again at RT for 5 m, and after another 2 m, centrifuged at 14000 g at 4° C. for 15 m. The top aqueous phase will be collected, mixed with equal volume of 75% ethanol, and processed on PureLink™ RNA spin columns (Invitrogen® on a QIAvac™ for 24 Plus apparatus (Qiagen®, Valencia, Calif.) as per manufacturer's protocols. RNA will be eluted from spin columns using 100 μL water, and stored at −80° C. until further use.

Chilled TRIzol Reagent containing 200 μL BAN and 10 μL of polyacryl carrier may be used with some samples to improve RNA recovery and yields. Optionally, bromoanisole may also be added to the mixture during this phase separation to improve the visualization of the isolated RNA and to remove chloroform and bromochloropropane from the isolation protocol.

The RNA containing the miRNA from other samples will be quantified using NanoDrop spectrophotometry or the Agilent quantification method. The concentration and integrity of the miRNA in at least some samples will be confirmed using the RNA 6000 nano LabChip Series II Assay with an Agilent Bioanalyzer.

The RNA will be reverse transcribed using stem loop RT primers, specific for each miRNA target and diluted with nuclease free water to give 50 μM concentration per reaction, according to standard terms and conditions. The DNA may then be stored at about −20° C. until further use. The relative quantification of the miRNA express levels may be conducted by real-time PCR, using the expression level of miR-16 and/or another stably expressed small RNA(s) to normalize the expression level of the target miRNA. All reactions will be performed in triplicate and using an interassay control. The data may be analyzed using 2-delta deltaCT to determine the relative quantities of the target miRNA.

For some samples, levels of mature miRNAs will be measured using TaqMan™ miRNA assays (Applied Biosystems®, foster City, Calif.). TaqMan miRNA reverse transcription kit will be used for reverse transcribing 9.9 μL RNA in 15 μL at 42° C. for 30 m using a miRNA-specific oligonucleotide. MiRNA-specific primers and 1.33 μL of RT reactions will be used in triplicate 40- or 42-cycle quantitative PCR, and SDSTM software (version 2.3, Applied Biosystems®) will be used to identify quantification cycle (C_{q}) values as the average values obtained from the triplicate PCR reactions.

The target miRNA circulating in the intraductal fluid sample that will be screened are listed in Table 3, Table 4, and Table 5. For e.g., circulating miR-195 is a marker for early stage breast cancer as well as myocardial infarction (MI) (Long et al. PLOS ONE. 2012, vol. 7(12) e50926). Increased miR-195 levels in blood are observed in breast cancer patients and MI. As another example, high miR-26a is associated with decreased EZH2 expression and with favorable outcome on tamoxifen in metastatic breast cancer (Jansen et al. Breast Cancer Res. Treat. 2012, 133:937-947). However, circulating miR-195 levels or miR26a in NAF are not known. Circulating miRNA-195 and miR-26a in NAF will be determined.

Statistical analyses and graphical plotting will be done using Prism™ (GraphPad software, La Jolla Calif.) and excel software (Microsoft). All t and Mann-Whitney U tests will be 2-tailed tests.

One of skill in the art will readily recognize the scope of the present invention to include the methods disclosed herein comprising screening for miRNAs and the ability to predict an individual's response to treatment with drugs such as tamoxifen and potential for side effects such as MI as a result of treatment with tamoxifen based on miRNA expression and signatures.

### Example 5

### Detection of DNA Methylation Signatures in Intraductal Fluid Sample

Intraductal fluid sample will be aspirated from the nipples of both breasts of a female subject undergoing mammography using a collection device comprising a breast engaging member which attaches the device to the breast. The intraductal fluid sample will be collected onto a solid phase sample collection medium such as an absorbent paper. The intraductal fluid sample absorbed on the absorbent paper will be washed and the DNA from the wash effluent will be extracted using Qiagen-DNAeasy Blood & Tissue Kit® according to the supplier's instructions (Qiagen, Valencia, Calif.). DNA will be extracted with QIAamp DNA Mini Kit® (Qiagen) by following the manufacturer's instructions. DNA will be quantitated using NanoDrop ND-1000 UV- is Spectrophotometer (NanoDrop® Technologies, Wilmington, Del.). Site-specific CpG methylation will be analyzed using Illumina Infinium® HumanMethylation 27 Bead Chips based technique (Illumina). This array was developed to assay 27,578 CpG sites selected from more than 14,000 genes. This will allow interrogation of all sites per sample at a single nucleotide resolution. Genomic DNA will be treated with sodium bisulfite using the Zymo EX DNA Methylation Kit (Zymo Research, Orange, Calif.) according to the manufacturer's protocol and chip processing and data analysis will be carried out using manufacturere's protocol. The quality of the bead array data will be checked with the GenomeStudio® Methylation Module software.

The foregoing description of the specific embodiments will so fully reveal the general nature of the invention that others can, by applying the current knowledge, readily modify and/or adapt for various applications such specific embodiments without undue experimentation and without departing from the generic concept, and therefore, such adaptation and modification should and are intended to be comprehended within the meaning and range of equivalents of the disclosed embodiments. Although the invention has been described in conjunction with specific embodiments thereof, it is evident that many alternatives, modifications, and variations will be readily apparent to one of skill in the art. Accordingly, it is intended to embrace all such alternatives, modifications and variations that fall within the spirit and broad scope of the appended claims.

It should be understood that the detailed description and specific examples are given by way of illustration only, since various changes and modifications within the spirit and scope of the invention will be apparent to one of skill in the art from this detailed description. One of skill in the art will also appreciate that certain features of the invention, which for clarity although described in the context of separate embodiments, may also be provided in combination in a single embodiment. Conversely, various features of the invention which for the sake of brevity, are described in a single embodiment, may also be provided separately or in any suitable sub-combination.

What is claimed is:

1. A method of diagnosing or prognosing a breast disorder in an individual in need thereof, comprising screening intraductal fluid obtained from a nipple of the individual during mammography for at least one biomarker associated with a breast disorder.

2. The method of claim 1, further comprising contacting the nipple with a collection device.

3. The method of claim 2, wherein the collection device comprises a solid phase sample collection medium.

4. The method of claim 3, wherein the collection device further comprises a breast engaging member which attaches the device to the breast.

5. The method of claim 4, wherein the solid phase sample collection medium is selected from absorbent paper, microscopic glass slides, capillary tubes, collection tubes, columns, micro-columns, wells, plates, membranes, filters, resins, inorganic matrices, beads, particulate chromatographic media, plastic microparticles, latex particles, coated tubes, coated templates, coated beads, coated matrices, or a combination thereof.

6. The method of claim 1, further comprising removing keratin from nipple ducts of the breast of the individual prior to the mammography.

7. The method of claim 1, further comprising administering atropine to the nipple of the individual prior to the mammography.

8. The method of claim 1, further comprising administering oxytocin to the individual prior to performing mammography.

9. The method of claim 1, wherein the at least one biomarker associated with a breast disorder comprises cytology, proteins, glycoproteins, DNA, RNA, gene mutations, single nucleotide polymorphism, DNA copy numbers, DNA methylation, histone methylation, miRNA, microbiome or a combination thereof.

10. The method of claim 1, wherein the screening comprises contacting a cell from the intraductal fluid with an antibody that binds to an antigen selected from the group consisting of: CK5, CK14, CK7, CK18, and p63.

11. The method of claim 1, wherein the screening comprises determining a presence and/or a level of one or more miRNA, profiling miRNA signature, or a combination thereof in the intraductal fluid sample.

12. The method of claim 9, wherein the miRNA is selected from Table 3, Table 4, Table 5, or a combination thereof.

13. The method of claim 9, wherein the miRNA in the intraductal fluid sample is exosomal.

14. The method of claim 11, wherein the screening of miRNA comprises amplification, sequencing, restriction length polymorphism analysis, microarray analysis, multiplex analysis, or a combination thereof.

15. The method of claim 14, wherein the amplification is performed by ligase chain reaction, polymerase chain reaction PCR, reverse-transcriptase PCR, quantitative PCR, real-time PCR, isothermal PCR, multiplex-PCR, or methylation-specific PCR.

16. The method of claim 14, wherein the sequencing is selected from the group consisting of dideoxy sequencing, reverse-termination sequencing, next generation sequencing, barcode sequencing, paired-end sequencing, pyrosequencing, deep sequencing, sequencing-by-synthesis, sequencing-by-hybridization, sequencing-by-ligation, single-molecule sequencing, single molecule real-time sequencing-by-synthesis, bisulfite-sequencing, whole genome sequencing, whole exome sequencing, RNA-seq, Whole Transcriptiome Shotgun Sequencing, transcriptome sequencing, and mRNA-Seq.

17. A method for classifying an individual as having a breast disorder comprising:
a) obtaining intraductal fluid from a nipple of the individual during mammography;
b) detecting a presence of at least one miRNA selected from Table 3, Table 4, Table 5, or a combination thereof in the intraductal fluid sample; and
c) classifying the individual as having a breast disorder if the detected miRNA has a measured value above said threshold value;
wherein the presence of the miRNA is detected when said miRNA has a measured value above a threshold value for the miRNA.

18. A method for classifying an individual as having a breast disorder comprising:
a) obtaining intraductal fluid from a nipple of the individual during mammography;
b) detecting a decreased presence of at least one miRNA selected from Table 3, Table 4, Table 5, or a combination thereof in the intraductal fluid sample; and
c) classifying the individual as having a breast disorder if the detected miRNA has a measured value below said threshold value;
wherein the decreased presence of the miRNA is detected when said miRNA has a measured value below a threshold value for the miRNA.

19. A method for classifying an individual as at risk for or having CCH, ADH, DCIS or IDC comprising:
a) obtaining intraductal fluid from a nipple of the individual during mammography; and
b) detecting an altered expression of at least one miRNA selected from Table 3.

20. The method of claim 1, wherein the screening comprises:
a) contacting a cell from the intraductal fluid with antibodies that bind to uPA, PAI-1, and Gal-GalNAc;
b) detecting in the intraductal fluid sample altered miRNA signature disclosed in Table 5; or
c) detecting in the intraductal fluid sample an altered DNA methylation pattern of uPA, PAI-1 and GalNac Transferases genes.

21. The method of claim 1, wherein the individual has a BI-RADS III or a BI-RADS IV lesion.

22. The method of claim 1, further comprising determining or modifying a treatment regimen for the individual based on results of the screening.

23. The method of claim 22, wherein the treatment regimen comprises a therapeutic agent, radiation therapy, and/or surgical excision of breast tissue.

24. The method of claim 23, wherein the therapeutic agent is an anthracycline, a platinum agent, a taxane, or a combination thereof.

25. The method of claim 23, wherein the therapeutic agent is ado-trastuzumab emtansine, albumin-bound paclitaxel, anastrozole, butyric acid, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen, tamoxifen derivative, 4-hydroxytamoxifen, N-desmethyltamoxifen, endoxyfen, cis-tamoxifen, toremifene, trastuzumab, vinorelbine, or a combination thereof.

26. The method of claim 23, wherein the therapeutic agent is a SERM, a SERD, an AI, a pharmaceutical salt thereof, or a combination thereof.

27. The method of claim 26, wherein the SERM is selected from the group consisting of tamoxifen, cis-tamoxifen, 4-hydroxytamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652, and ERA-92.

28. The method of claim 26, wherein the therapeutic agent further comprises at least one omega-3 fatty acid and at least one vitamin D compound.

29. A method of diagnosing or prognosing a breast disorder in an individual having a BI-RADS III or a BI-RADS IV lesion, comprising screening intraductal fluid sample obtained from a nipple of the individual during mammography for at least one biomarker associated with a breast disorder.

30. The method of claim 29, comprising contacting the nipple with a collection device.

31. The method of claim 30, wherein the collection device comprises a solid phase sample collection medium.

32. The method of claim 31, wherein the collection device further comprises a breast engaging member which attaches the device to the breast.

33. The method of claim 31, wherein the solid phase sample collection medium is selected from absorbent paper, microscopic glass slides, capillary tubes, collection tubes, columns, micro-columns, wells, plates, membranes, filters, resins, inorganic matrices, beads, particulate chromatographic media, plastic microparticles, latex particles, coated tubes, coated templates, coated beads, coated matrices, or a combination thereof.

34. The method of claim 29, further comprising removing keratin from nipple ducts of the breast of the individual prior to the mammography.

35. The method of claim 29, further comprising administering atropine to the nipple of the individual prior to the mammography.

36. The method of claim 29, further comprising administering oxytocin to the individual prior to performing mammography.

37. The method of claim 29, wherein the biomarker associated with a breast disorder comprises cytology, proteins, glycoproteins, DNA, RNA, gene mutations, single nucleotide polymorphism, DNA copy numbers, DNA methylation, histone methylation, miRNA, microbiome or a combination thereof.

38. The method of claim 29, wherein the screening comprises contacting a cell from the intraductal fluid with an antibody that binds to an antigen selected from the group consisting of: CK5, CK14, CK7, CK18, and p63.

39. The method of claim 29, wherein the screening comprises determining a presence and/or a level of one or more miRNA, profiling miRNA signature, or a combination thereof in the intraductal fluid sample.

40. The method of claim 39, wherein the miRNA is selected from Table 3, Table 4, Table 5, or a combination thereof.

41. The method of claim 40, wherein the miRNA in the intraductal fluid sample is exosomal.

42. The method of claim 39, wherein the screening comprises miRNA amplification, sequencing, restriction length polymorphism analysis, microarray analysis, multiplex analysis, or a combination thereof.

43. The method of claim 42, wherein the amplification is performed by ligase chain reaction, polymerase chain reaction, reverse-transcriptase PCR, quantitative PCR, real-time PCR, isothermal PCR, multiplex-PCR, or methylation-specific PCR.

44. The method of claim 42, wherein the sequencing is selected from the group consisting of dideoxy sequencing, reverse-termination sequencing, next generation sequencing, barcode sequencing, paired-end sequencing, pyrosequencing, deep sequencing, sequencing-by-synthesis, sequencing-by-hybridization, sequencing-by-ligation, single-molecule sequencing, single molecule real-time sequencing-by-synthesis, bisulfite-sequencing, whole genome sequencing, whole exome sequencing, RNA-seq, Whole Transcriptiome Shotgun Sequencing, transcriptome sequencing, and mRNA-Seq.

45. A method for classifying an individual having a BI-RADS III or a BI-RADS IV lesion as having a breast disorder comprising:
a) obtaining intraductal fluid from a nipple of the individual during mammography;
b) detecting a presence of at least one miRNA selected from Table 3, Table 4, Table 5, or a combination thereof in the intraductal fluid sample; and
c) classifying the individual as having a breast disorder if the detected miRNA has a measured value above said threshold value;
wherein the presence of the miRNA is detected when said miRNA has a measured value above a threshold value for the miRNA.

46. A method for classifying an individual having a BI-RADS III or a BI-RADS IV lesion as having a breast disorder comprising:
a) obtaining intraductal fluid from a nipple of the individual during mammography;
b) detecting a decreased presence of at least one miRNA selected from Table 3, Table 4, Table 5, or a combination thereof in the intraductal fluid sample; and
c) classifying the individual as having a breast disorder if the detected miRNA has a measured value below said threshold value;
wherein the decreased presence of the miRNA is detected when said miRNA has a measured value below a threshold value for the miRNA.

47. A method for classifying an individual having a BI-RADS III or a BI-RADS IV lesion as at risk for or having CCH, ADH, DCIS or IDC comprising:
a) obtaining intraductal fluid from a nipple of the individual during mammography; and
b) detecting an altered expression of at least one miRNA selected from Table 3.

48. The method of claim 29, wherein the screening comprises:
a) contacting a cell from the intraductal fluid with antibodies that bind to uPA, PAI-1, and Gal-GalNAc;
b) detecting in the intraductal fluid sample altered miRNA signature disclosed in Table 5; or
c) detecting in the intraductal fluid sample an altered DNA methylation pattern of uPA, PAI-1 and GalNac Transferases genes.

49. The method of claim 29, further comprising determining or modifying a treatment regimen for the individual based on results of the screening.

50. The method of claim 49, wherein the treatment regimen comprises a therapeutic agent, radiation therapy, and/or surgical excision of breast tissue.

51. The method of claim 50, wherein the therapeutic agent is an anthracycline, a platinum agent, a taxane, or a combination thereof.

52. The method of claim 50, wherein the therapeutic agent is ado-trastuzumab emtansine, albumin-bound paclitaxel, anastrozole, butyric acid, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen, 4-hydroxytamoxifen, N-desmethyltamoxifen, endoxifen, cis-tamoxifen, toremifene, trastuzumab, vinorelbine, or a combination thereof.

53. The method of claim 50, wherein the therapeutic agent is a SERM, a SERD, an AI, a pharmaceutical salt thereof, or a combination thereof.

54. The method of claim 53, wherein the SERM is selected from the group consisting of tamoxifen, cis-tamoxifen, 4-hydroxytamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-92.

55. The method of claim 53, wherein the therapeutic agent further comprises at least one omega-3 fatty acid and at least one vitamin D compound.
